(19) United States
(12) Patent Application Publication
(10) Pub. No.: US 2008/0050815 A1
(43) Pub. Date: Feb. 28, 2008

(54) Method of oocyte cryopreservation including piercing the zona pellucida prior to vitrification

(76) Inventors: Geoffery Sher, Las Vegas, NV (US); Levent Keskintepe, Las Vegas, NV (US)

Correspondence Address: MCDERMOTT WILL & EMERY LLP, 600 13TH STREET, N.W., WASHINGTON, DC, 20005-3096, US

(21) Appl. No.: 11/826,998

(22) Filed: Jul. 19, 2007

Related U.S. Application Data
(60) Provisional application No. 60/831,687, filed on Jul. 19, 2006.

(51) Int. Cl. C12N 5/06

(52) U.S. Cl. 435/374

(57) ABSTRACT

The present invention relates to the cryopreservation and preparation of warmed oocytes for fertilization. Cryopreservation of oocytes using vitrification methods is disclosed. Exemplary techniques include making a vent in the zona pellucida surrounding the oocyte; contacting the oocyte with a vitrification solution comprising at least one cryoprotectant; and vitrifying the oocyte. Further methods of warming the vitrified oocyte, in preparation for intracellular sperm injection are disclosed, allowing for the assessment of successful fertilization.

### BACKGROUND OF THE INVENTION

1. Field of the Invention

The present invention relates to methods of cyropreservation. More specifically, it relates to methods of improving oocyte cyropreservation.

2. Background

Cryopreservation refers to the process of cooling and storing cells, tissues, or organs at very low temperatures to maintain their viability. Until recently, cryopreservation allowed water to crystallize, disrupting cellular membranes, due to conventional freezing techniques. Additionally, as water crystallized, concentrations of solutes could increase to toxic levels. The resulting damage would be apparent upon thawing.

The recent introduction of vitrification to cryopreservation methods as a rapid cooling technique promises to avoid the cellular damage and toxicity problems caused by conventional freezing. Rather than solidifying a sample by ice crystallization, vitrification involves the solidification of a sample by greatly increasing the sample's viscosity as the sample is plunged into liquid nitrogen.

Vitrification has been used in Assisted Reproductive Technology as a cryopreservation method for gametes, early embryos, blastocysts and ovarian tissue. Successful vitrification of mouse embryos was first reported in 1985. The technique was then further applied and improved in animal reproduction. The greatest advantage of vitrification over conventional freezing techniques has been seen in oocytes and blastocysts, which are extremely sensitive to ice crystallization.

The first successful human pregnancies following oocyte vitrification were reported in 1999 and 2000. Since then, growing scientific interest in oocyte vitrification has spawned considerable research for more effective and practicable techniques. This new scientific interest could herald a shift from embryo to oocyte cryopreservation, leading to wide-scale egg banking.

One advancement in vitrification techniques for blastocysts has involved decreasing the volume of blastocoelic fluid present. Studies have reported that creating an opening in a blastocyst's zona pellucida allows blastocoelic fluid to escape, and hence decreases the amount of fluid at risk for ice crystallization. The resulting survival and pregnancy rates for blastocysts with opened zona pellucidas are significantly higher than those rates for blastocysts with intact zona pellucidas (Zech et al., Reprod Biomed Online. September 2005; 11(3): 355-61). However, it had not been determined if opening the zona pellucida of oocytes prior to vitrification increases the oocytes' cryosurvival rate.

The structure and characteristics of mammalian oocytes pose unique challenges. The oocyte is one of the largest cells in the mammalian body, and due to its spherical shape has an unusually low surface area-to-volume ratio compared to other cell types (Smith et al., Reprod Biomed Online. August 2004; 9(2): 171-8; Wright et al., Reprod Biomed Online. August 2004; 9(2): 179-86). Morphological characteristics of oocytes such as maturity and size, and biophysical factors such the cells' response to certain cryoprotectant compositions are particularly important considerations in developing cryopreservation methods. Special attention must be paid to the oocyte's exquisite sensitivity to ice crystal formation, which can yield a variety of cellular injuries including the formation of damage intracellular lipid droplets and to the cytoskeleton. Furthermore, the low permeability of oocyte cytoplasmic membranes leads to high susceptibility to osmotic swelling during the removal of cryoprotectant. These challenges are present in all cryopreservation methods, but may be better addressed by vitrification than by conventional freezing techniques.

Recent studies indicate that faster cooling times increase oocyte cryosurvival rates. A study conducted by Liebermann et al., (Methods Mol Biol. 2004; 254: 345-64), utilized very rapid cooling of 11.5 minutes and rapid warming of 15 minutes. These times are considerably shorter than those of the conventional freezing cryopreservation protocol reported by Fabbri et al., (Hum Reprod. March 2001; 16(3):411-6), which requires 108 minutes for freezing and 30 minutes for warming. Liebermann et al. concluded that a rapid cooling rate is important in order to improve the effectiveness of oocyte vitrification. However, in spite of Liebermann et al. and others' advances, there remain significant technical problems associated with oocyte cryopreservation. Improvements to vitrification techniques would include the further reduction of cooling and warming times, of incidence of ice crystallization, and of cryoprotectants' toxicity to cells.

Oocyte vitrification is still not widely used by in vitro fertilization (IVF) laboratories and as yet no standardized vitrification protocol has been defined. There are many variables inherent in vitrification protocols and the cryosurvival rates of cells and tissues have thus far been inconsistent. Appropriate cryoprotectants need to be determined as well as methods of increasing oocyte permeability to these to decrease cooling times. There currently exists a need to develop a method of vitrifying oocytes which is both effective and practicable.

The invention provides these and other advantages, as will be apparent to those skilled in the art based on the disclosure hereunder. All references and documents cited herein are incorporated by reference in their entirety.

### SUMMARY OF THE INVENTION

The present invention is directed toward a method of oocyte cryopreservation comprising obtaining an oocyte; making at least one vent in the zona pellucida surrounding the oocyte; contacting the oocyte with a vitrification solution comprising at least one cryoprotectant; and vitrifying said oocyte.

In one embodiment, the vent is a slit about 10 μm to about 15 μm in length.

In a preferred embodiment, the vent is made by laser incision, such as by a 1.48 micron infrared diode laser. In an alternative embodiment, the vent is made by piercing the zona pellucida with a sharp pipette or microneedle. In another embodiment, the vent is made by microscopically applying acidified media, such as acidified tyrode solution, to a focal area of the zona pellucida to induce local thinning.

In a preferred embodiment, the steps of obtaining an oocyte, making at least one vent in the zona pellucida surrounding the oocyte, and contacting the oocyte with a vitrification solution, are all carried out at a temperature of about 36° C. to about 38° C.

In a preferred embodiment, prior to vitrification, the oocyte is treated with a vitrification solution. Preferably, the oocyte is treated with a series of vitrification solutions with sequentially increasing concentrations of cryoprotectants. In one embodiment, the cryoprotectants of the vitrification solutions in the series are ethylene glycol (EG) and dimethylsulphoxide (DMSO). Most preferably, the cryoprotectant concentrations in the series of vitrification solutions range from about 0.625% to about 20.000%.

In a preferred embodiment, the oocyte is vitrified by directly plunging the oocyte into liquid nitrogen. In another embodiment, the oocyte is loaded onto a cryoloop before being plunged into the liquid nitrogen.

In yet a further embodiment, following vitrification, the oocyte is treated with a series of warming solutions comprising sequentially decreasing concentrations of non-permeating cryoprotectant, such as sucrose. Most preferably, the non-permeating cryoprotectant concentrations in the series of warming solutions range from about 0.0 M to about 1.5 M.

In a further embodiment, after treatment with warming solutions, the oocyte is washed and cultured before a step of fertilization by intracytoplasmic sperm injection (ICSI).

The present invention yielded an oocyte viability rate of 90%, whereas conventional occyte freezing techniques yield a viability rate of only 70%.

Additional advantages of the present invention will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention. The present invention may be practiced without some or all of these specific details. In other instances, well known process operations have not been described in detail, in order not to unnecessarily obscure the present invention.

### DETAILED DESCRIPTION OF THE INVENTION

It is an object of the invention to provide methods of vitrification of oocytes. Additionally, the invention allows for assessment of oocyte viability at least two hours after warming, and allows for assessment of successful fertilization of warmed oocytes within at least eighteen hours of intracellular sperm injection (ICSI).

Prior to describing the invention in more detail, the following definitions are intended for the terms used herein:

The term “vitrification” as used herein refers to the solidification of a biological sample at low temperature, not by ice crystallization but by extreme elevation in viscosity during cooling. Preferably, an oocyte is suspended at about 0° C. or above, in a highly concentrated solution of permeating cryoprotectant, which largely displaces water in the oocyte. The oocyte may then be loaded onto a cryoloop before plunging it directly into liquid nitrogen and achieving a cooling of between 15,000 to 30,000° C./min. Water is transformed directly from the liquid phase to a glassy, vitrified state. With this method fewer damaging ice crystal form than in conventional freezing. Although the terms “freezing” and “thawing” are commonly used for conventional cryopreservation, the terms “cooling” and “warming” are used herein for vitrification procedures. Vitrification protocols are constantly being improved and are simple, fast, and inexpensive.

The term “vitrification solution” as used herein shall mean any aqueous mixture containing at least one permeating cryoprotectant. The oocyte is preferably contacted with a series of vitrification solutions with cryoprotectants of increasing concentrations prior to vitrification. Preferably, permeating cryoprotectants are added to an initial vitrification solution comprising G-MOP, (HEPES buffered amino acid produced by In vitro life).

enhanced media and 5 mg human serum albumin (HSA)/ml. Preferably, permeating cryoprotectant concentrations in vitrification solution range from about 0% to less than or equal to about 20%. Most preferably, the combined cryoprotectant concentration in each solution of a series of vitrification solutions is about 0.625%, about 1.25%, about 2.5%, about 5%, about 10%, and about 20%.

There exist special carrier systems for vitrification solutions (e.g., the open pulled straws Medical Technology (MTG), Vertriebs-GmbH, Germany, the flexipet-denuding pipette FDP Cook IVF, Spencer, Ind., the cryoloop Hampton Research, Laguna Niguel, Calif.), or the cryostraw for the hemistraw system IVM, L'Aigle, France), all of which minimize the volume of the vitrification solution to keep the cooling rate as high as possible.

In one embodiment of the claimed method, all steps other than vitrification are carried out at about 36° C. to about 38° C. In the preferred embodiment, all steps other than vitrification are carried out at about 37° C.

The term “cryoprotectant” as used herein refers to a substance that is used to protect a cellular sample from damage due to ice crystallization.

The term “permeating cryoprotectant” as used herein shall mean a cryoprotectant useful in the present invention capable of penetrating the cellular plasma membrane. Permeating cryoprotectants include but are not limited to dimethylsulphoxide (DMSO), acetamide, and 1,2 propanediol, for example. Additional permeating cryoprotectants include but are not limited to glycerol and glycols such as ethylene glycol (EG) and propylene glycol. Prefereably, osmotic pressures inside and outside the cell increase equally with the concentration of permeating cryoprotectants. Most prefereably, a permeating cryoprotectant can serve as both a solvent and a solute in a vitrification solution.

The term “non-permeating cryoprotectant” shall mean a cryoprotectant which does not penetrate cellular plasma membranes. Non-permeating agents facilitate the speed at which vitrification occurs and also mitigate cellular damage. Prefereably, high concentrations of non-permeating cryoprotectants in vitrification solutions draw water out of the oocyte by osmosis. A decreased volume of water in the oocyte reduces the volume of permeating cryoprotectants required to completely displace water from the oocyte, and shortens the time required for the displacement to be completed. Additionally, non-permeating cryoprotectants are included in warming solutions to decrease swelling of the oocyte as permeating cryoprotectants are removed from the cell. Non-permeating cryoprotectants include but are not limited to macromolecules and sugars. Prefereably, non-permeating cryoprotectants include sucrose, fructose, glactose, lactose, mannose, raffinose and trehalose; proteins found in milk and egg yolk, such as albumin; amides; synthetic polymers such as Ficoll, polyethylene glycol, polyvinylpyrrolidone or methyl cellulose; and algae-derived polysaccharides such as agarose and alginate. Non-permeating cryoprotectants typically do not serve as solvents.

The term “warming solution” as used herein shall mean any aqueous mixture applied to the oocyte after vitrification. The oocyte is preferably contacted with a series of warming solutions after vitrification of sequentially decreasing concentrations of non-permeating cryoprotectant. In one embodiment, the non-permeating cryoprotectant is added to an initial warming solution comprising G-MOP and 5 mg human serum albumin (HSA)/ml. Preferably, concentrations of the non-permeating cryoprotectant in the series ranges from about 0 M to about 1.5 M. Most preferably, the non-permeating cryoprotectants are sucrose and Ficoll. Preferably, the sucrose concentration in each warming solution of the series is about 1.5 M, about 0.75 M, about 0.5 M, about 0.25 M, about 0.125 M, and about 0 M.

The term “oocyte” as used herein refers to an female gamete. The oocyte is a large and essentially stationary cell which undergoes meioses when fertilized by a sperm, the male gametocyte. Oocytes are classified as primary or secondary oocytes, depending on whether they have undergone zero or one meiotic divisions. Two primary oocytes are created when an oognium divides by mitosis. Each primary oocyte then divides in meiosis I into a haploid secondary oocyte, and typically a first polar body with less cytoplam than the secondary oocyte, which soon disintegrates. However, cytoplamic distribution can be equal and result in two secondary oocytes. A second meiosis then occurs. The oocytes described herein are prefereably in the metaphase stage of this second meiotic division. Preferably, a sample oocyte is obtained a prepared for cryopreservation through methods well-known in the art.

The term “zona pellucida” as used herein refers to the glycoprotein membrane surrounding the plasma membrane of an oocyte or blastocysts. Surrounding and penetrating the zona pellucida is the corona radiata, a single layer of columnar granulose cells that assist in both providing nutrients to the oocyte and in regulating the maturation of the oocyte. Several more layers of granulose cells may surround the corona radiata.

The term “vent” as used herein shall mean an opening in the zona pellucida surrounding an oocyte or blastula. Preferably a vent shall mean a slit opening of about 5 μm to about 25 μm in length, more preferably between about 10 μm to about 20 μm, and about 10 μm to about 15 μm and most preferably between about 12 μm to 15 μm in length. The vent shall preferably penetrate all layers of the zona pellucida, but not the oocyte's plasma membrane. Preferably a vent is made by laser incision, using for example, a 1.48 micron infrared diode laser. A vent may also be made by piercing the zona pellucida with a sharp pipette or microneedle. Alternatively, a vent may be made by microscopically applying acidified media, such as tyrode solution, to the zona pellucida to induce local thinning. Preferably, at least one vent is made in the zona pellucida. Most preferably, one vent is made in the zona pellucida.

Following creation of a vent in the zona pellucida surrounding the oocyte, the oocyte is preferably contacted with a vitrification solution and promptly vitrified. Following vitrification, the oocyte may be treated with a series of warming solutions containing non-permeating cryoprotectants of sequentially decreasing concentrations. The oocytes may then be washed and cultured before fertilization by intracytoplasmic sperm injection (ICSI), for example.

The term “cryosurvival rate” refers to the number of oocytes that are morphologically intact post-thawing/warming, as a percentage of the total number cryopreserved.

The terms “a” or “the” shall refer to either the plural or the singular form of the referenced noun.

Vitrification protocols presented herein will be applicable to egg banking. Egg banking can be used for the purpose of fertility preservation (FP) to allow for family planning, oocyte donation, or to preserve fertility prior to ovarian failure. Egg banking may also be a desirable option for women of reproductive age with malignant diseases, the treatment of which involves surgery, radiation, or chemotherapy, which would lead to oocyte destruction. Egg banking may also be used by persons with moral or religious objections to embryo but not oocyte cryopreservation.

Vitrification Protocol

In one preferable embodiment, non-permeating cryoprotectants are present in the vitrification solution. Non-permeating cryoprotectants promote the rate of vitrification and decrease the risk of cellular damage. Non-permeating cryoprotectants include, for example, macromolecules and sacharides. Specific non-permeating agents include sucrose, fructose, galactose, lactose, mannose, raffinose and trehalose; proteins, such as those found egg yolk or milk, including for example albumin or bovine serum; synthetic polymers such as polyethylene glycol, polyvinylpyrrolidone, methyl cellulose or amides. Algae-derived polysaccharides such as agarose and alginate are useful as non-permeating cryoprotectants. Most preferably, sucrose is used. Inclusion of a non-permeating cryoprotectant preferably also helps prevent over-swelling of the cell during the warming process when permeated cryoprotectant is removed.

### EXAMPLE 1

In the following example, all steps other than vitrification were carried out at 37° C. First, cumulus cells were removed from a sample cumulus-oocyte complex by gentle pipetting. The oocyte was then transferred into human tubal fluid (HTF) supplemented with 10% synthetic serum for two hours. After incubation for 2 hours, a 10 μm to 15 μm vent was made in the zona pellucida using a Becton Dickinson laser.

Five minutes after the vent was created, the oocyte was treated with vitrification solutions, in sequentially increasing concentrations of permeating cryoprotectants. The initial vitrification solution was prepared by combining G-MOP with 5 mg HSA/ml. Then, 8 parts of the initial vitrification solution were combined with 1 part ethylene glycol (EG) and 1 part dimethylsulphoxide (DMSO). These concentrations such that the EG and DMSO comprised 0.625% of the solution. The oocyte was then submersed in the vitrification solution comprised of 0.625% dimethylsulphoxide (DMSO) and ethylene glycol (EG). After that, the oocyte was moved into a solution comprised of 1.25% DMSO and EG for 1 minute. Next, the oocyte was moved into a solution comprising 10% DMSO and EG for 1 minute. Finally, the oocyte was moved into a solution comprising 20% DMSO and EG, and held there for only 20 seconds.

While in the 20% DMSO and EG solution, the oocyte was loaded onto a cryoloop and directly plunged into liquid nitrogen. After the vitrification in liquid nitrogen, the oocyte was stored at about −190° C. for 1-3 months.

The first step in removing the oocyte from cryopreservation was to warm the oocyte directly plunging the cryoloop into a series of warming solutions held at about 37° C. with sequentially decreasing concentrations of sucrose. First the oocyte was plunged into a warming solution with 1.5 M sucrose for 50 seconds. After that, the oocyte was moved into a 0.75 M sucrose solution for 15 seconds. Next, the oocyte was held in 0.5 M sucrose solution for one minute. Then the oocyte was submersed in 0.25 M sucrose for one minute. After that, the oocyte was moved into a 0.125 M sucrose for one minute.

The warmed oocyte was then washed three times with global one media after the last warming solution. The oocyte was then cultured in global one media for about 2 hours before ICSI.

### EXAMPLE 2

An alternative approach to the oocyte vitrification process utilized the same methods as disclosed in Example 1, except for those steps described differently below. The initial vitrification solution was prepared by combining G-MOP with 5 mg HSA/ml. Next, the oocyte was submersed in the vitrification solution of 1.25% ethylene glycol (EG) for 1 minute and then moved into a solution of 2.5% EG for 1 minute. Following that, the oocyte was transferred into a solution of 5% DMSO and EG for 1 minute and thereupon is moved into a solution of 20% EG for 1 min. Finally, the oocyte was transferred into a solution of 40% EG, and held there for only 20 seconds. While in the 40% EG solution, the oocyte was loaded onto a cryoloop, directly plunged into liquid nitrogen and then cryo-stored at about −190° C.

While different embodiments of the invention have been described in detail herein, it will be appreciated by those skilled in the art that various modifications and alternatives to the embodiments could be developed in light of the overall teachings of the disclosure. Accordingly, the particular methods and arrangements are illustrative only and are not limiting as to the scope of the invention that is to be given the full breadth of any an all equivalents thereof.

### EXAMPLE 3

Results achieved using the methods of the present invention were compared with the results compared with conventional methods. Results using conventional methods were obtained from Barritt et al., Donor oocyte cryopreservation resulting in high pregnancy and implantation rates. Fertility and Sterility 86 (Suppl 2) p-473, S311.

As shown in Table I, 16 female patients out of 20 were selected for this study using the methods of the present invention. Out of these 16 patients, 12 showed pregnancies based on BhGC levels, and 9 patients were still pregnant after 6-7 weeks, based on ultrasound testing. Based on these results, only 1.6 embryos were transferred per patient.

In contrast, the conventional method selected 5.75 embryos per patient. As such, on a patient per patient comparison, the results obtained using the instant methods are more successful than those obtained using conventional methods.

Eggs were frozen according to the methods herein, the eggs were then selected for thawing following polar body biopsy with comparative genomic hybridization.

Furthermore, Table I, shows the number of oocytes using the methods of the present invention and the number of oocytes using conventional methods. The number of oocytes that were thawed using the present methods, was 48, 46 or 96% of which survived vitrification. Of these 46 oocytes, 44 were successfully fertilized, ultimately resulting in 11 pregnancies, or 44% as indicated by fetal heart beat.

In contrast, results using conventional methods show that of 79 thawed oocytes, 68, or 86% survived and 61 were fertilized, ultimately resulting in only 6 or 26% pregnancies.

A comparison of these results shows that the methods of oocyte vitrification of the present invention allow a greater rate of births based on a few number of embryos transferred per patient.

TABLE 1
Oocyte vitrifiction results comparison

Number (%)

| | | Study | Conventional |
|---|---|---|---|
| | | group | method |
| Number of | Number of Patients | 20 | 23 |
| patients | Number of patients | 16 (80%) | 4 |
| | w/Embryo transfer | | |
| | ˜Number of embryos | 1.6 | 5.75 |
| | transferred/patient | | |
| | Number + BhCG | 12 (75%) | Not given |
| | Number Ultrasound | 9 (57%) | 3 (75%) |
| | Number lost | 3 (25%) | Not given |

| | | Study | Conventional |
|---|---|---|---|
| | | group | method |
| Number of | Number of oocytes thawed | 48 | 79 |
| Oocytes | Number of oocytes survived | 46 (96%) | 68 (86%) |
| | Number oocytes fertilized | 44 (96%) | 61 (89%) |
| | Number Embryos transferred | 25 | 23 |
| | Number Fetal heart beats | 11 (44%) | 6 (26%) |
| | (FHB) | | |

What is claimed:

1. A method of oocyte cryopreservation comprising:
a) obtaining an oocyte from a female donor;
b) making at least one vent in the zona pellucida of the oocyte;
c) contacting the oocyte with a vitrification solution comprising at least one cryoprotectant; and
d) vitrifying said oocyte.

2. The method of claim 1, wherein the vent is a slit about 10 μm to about 15 μm in length.

3. The method of claim 1, wherein the vent is made by laser incision.

4. The method of claim 3, where a 1.48 micron infrared diode laser is used.

5. The method of claim 1, wherein the vent is made by piercing the zona pellucida with a sharp pipette.

6. The method of claim 1, wherein the vent is made by piercing the zona pellucida with a microneedle.

7. The method of claim 1, wherein the vent is made by microscopically applying acidified media to a focal area of the zona pellucida to induce local thinning.

8. The method of claim 7, wherein the acidified media is acidified tyrode solution.

9. The method of claim 1 wherein steps (a), (b), and (c) are carried out at a temperature from about 36° C. to about 38° C.

10. The method of claim 1, wherein prior to vitrification, the oocyte is treated with a series of vitrification solutions of sequentially increased concentrations of cryoprotectants.

11. The method of claim 10, wherein the cryoprotectants solutions are selected from the group consisting of ethylene glycol (EG) and dimethylsulphoxide (DMSO)

12. The method of claim 11 wherein the combined concentrations of the cryoprotectants range from about 0.625% to about 20.000%.

13. The method of claim 1, wherein step (d) comprises directly plunging the oocyte into liquid nitrogen.

14. The method of claim 13, wherein the oocyte is loaded onto a cryoloop before being plunged into the liquid nitrogen.

15. The method of claim 1, wherein following vitrification, the oocytes are treated with a series of warming solutions comprising sequentially decreased concentrations of non-permeating cryoprotectant at about 36° C. to about 38° C.

16. The method of claim 15 wherein the non-permeating cryoprotectant is sucrose.

17. The method of 15, wherein the concentrations of non-permeating cryoprotectant in warming solutions of the series range decrease sequentially from about 1.5 M to about 0.0 M.

18. The method of claim 15, wherein after treatment with warming solutions, oocytes are washed and cultured before a step of fertilization by intracytoplasmic sperm injection (ICSI).
